(19) United States
(12) Patent Application Publication
(10) Pub. No.: US 2022/0008745 A1
(43) Pub. Date: Jan. 13, 2022

(54) USE OF VIRTUAL REALITY, AUGMENTED REALITY AND/OR MIXED REALITY TO DIAGNOSE AND/OR TREAT SKIN CONDITIONS

(71) Applicant: Johnson & Johnson Consumer inc., Skilliman, NJ (US)

(72) Inventor: FRANK J. KIRCHNER, Edison, NJ (US)

(21) Appl. No.: 17/295,012

(22) PCT Filed: Nov. 18, 2019
(86) PCT No.: PCT/IB2019/059893

§ 371 Date: May 18, 2021

Related U.S. Application Data
(60) Provisional application No. 62/769,213, filed on Nov. 19, 2018.

(51) Int. Cl. A61N 5/06; G16H 20/70; G16H 40/63

(52) U.S. Cl. CPC A61N 5/0616; A61N 2005/0647; G16H 40/63; G16H 20/70

(57) ABSTRACT

Systems and methods for using virtual reality, augmented reality and/or mixed reality content for diagnosis and treatment of skin conditions are provided. The system may comprise a VR device for displaying the VR content to the user and one or more biometric monitors for monitoring the user's biometrics before, during and/or after exposure to the VR content. The system may further include a processor and one or more modules for analyzing the user's biometrics. The method may include the steps of measuring the user's initial biometric data, exposing the user to selected VR content, measuring the user's biometric data during and/or after exposure to the VR content, analyzing changes in the user's biometric data resulting from the selected VR content, and determining whether the selected VR content as a positive effect on the skin condition of the user.

### BACKGROUND OF THE INVENTION

The skin is the body's largest organ. It covers and protects the body. Skin:
maintains water and electrolyte balance;
is a physical barrier that protects underlying tissues from injury, UV light, and bacterial invasion;
helps with feeling, e.g., heat, cold, pain, pressure and touch;
perceives and informs the brain of changes in the environment;
maintains body temperature; and
assists with making vitamin D.

Medications used to treat skin conditions include topical and oral drugs. Some common topical treatments for skin conditions include: antibacterials, including mupirocin or clindamycin, which are often used to treat or prevent infection; anthralin, which helps reduce inflammation and can help treat psoriasis; antifungal agents, including clotrimazole (Lotrimin), ketoconazole (Nizoral), and terbinafine (Lamisil AT), which are topical antifungals; benzoyl peroxide and creams, gels, washes and foams containing benzoyl, which are used to treat ringworm and athlete's foot; peroxide, which is used to treat acne; coal tar, which is used to treat conditions including seborrheic dermatitis (usually in shampoos) or psoriasis; corticosteroids, which are used to treat skin conditions including eczema; crisaborole (Eucrisa), which is used to treat mild to moderate eczema; retinoids, including Retin-A and Tazorac, which are used to treat conditions including acne; salicylic acid, which is the active ingredient in many skin care products for the treatment of acne and warts. Some common oral or injection treatments for skin conditions include: oral antibiotics, including dicloxacillin, erythromycin and tetracycline; antifungal drugs, including fluconazole and itraconazole, which are used to treat more severe fungal infections; antiviral agents, including acyclovir (Zovirax), famciclovir (Famvir) and valacyclovir (Valtrex), which are used for skin conditions including those related to herpes and shingles; corticosteroids, including prednisone, which are used to treat skin conditions linked to autoimmune diseases including vasculitis and inflammatory diseases such as eczema; immunosuppressants, including azathioprine (Imuran) and methotrexate (Trexall), which are used to treat conditions including severe cases of psoriasis and eczema; biologics, including adalimumab (Humira), adalimumab-atto (Amjevita), a biosimilar to Humira, brodalumab, (Siliq), etanercept (Enbrel), etanerceptszzs, (Erelzi), a biosimilar to Enbrel, infliximab (Remicade), ixekizumab, (Taltz), secukinumab (Cosentyx) and ustekinumab (Stelara), which are the latest methods being utilized to treat psoriasis and other conditions; enzyme inhibitors, including apremilast (Otezla), which is used to treat inflammation, and Eucrisa, which is used to treat mild to moderate atopic dermatitis/eczema; retinoids, including Acetretin (Soriatane), which is specifically used to treat all types of severe psoriasis.

Accordingly, a need exists for new approaches in diagnosing and treating skin conditions.

### SUMMARY OF THE INVENTION

Virtual Reality (VR) exploits a psychological phenomenon known as “presence”—the illusion that you are really in an environment, not just looking at a picture of it. What we perceive as the real world is constructed by our brains. Information from the sensory organs is taken in by the brain and matched to previously stored information in memory to create the reality that we perceive and interact with. This “presence” tricks the brain into experiencing fake objects as if they're real ones and due to this phenomena research has suggested that there is a therapeutic potential of VR to target many areas of health, including in cognitive development, emotional regulation, cognitive function and other physiological issues.

Separately, current research suggests the occurrence of communication axes between organs, such as the brain-skin axis. Data from these studies support the observations in which response to stress can be correlated to skin inflammation, i.e., stressful life events may trigger and/or exacerbate inflammatory skin disease. The brain is directly responsible for communicating with the skin to initiate physiological cascades in response to certain sensory perception.

It is believed that the brain can be tricked to perceive “fake” sensation and communicate with distal organs via the Gut-Skin Axis. Essentially, it is believed that VR, AR and/or MR can be used to prove the existence of the communication axes between the brain and skin (Brain to Skin Axis [BSA]).

The experiment design would assess how VR, AR and MR could enable the existence of the BSA. This method could be, but would not be limited to, either an observational or clinical trial in which specific methods would be used to subject a VR/AR/MR simulation, experience and/or environment (associated with but not limited to sun exposure [i.e., beach setting with multiple electromagnetic radiation], pollution exposure, compromised skin diseases [i.e., acne, atopic dermatitis, eczema, psoriasis], aging skin and disorders, wounds, wound healing, scarring, etc.) to a human subject while simultaneously collecting specific biometrics (such as but not limited to brainwaves/brainwave activity, proinflammatory factors, chemoattractant factors, cytokines, chemokines, interleukins, lymphocytes, adhesion molecules, proteases, direct inflammatory cytokines, regulatory cells, pH, skin conductance, lipid concentration) before, during and after VR/AR/MR simulation.

Observed changes in collected biometrics after subjection to VR/AR/MR simulation/experience and/or environment when compared to baseline biometrics would increase confidence of the existence of the BSA.

An object of the invention is to utilize learnings from VR, AR and/or MR to help prevent and/or treat skin conditions resulting from eczema and other environmentally-induced skin adverse events.

The present invention generally fulfils at least some of these needs and also other needs which will be apparent to those skilled in the art upon reading the following specification.

The skin, as the largest and most visible organ of the body, is subject to constant exposure to a wide range of materials. These materials may be natural or synthetic and can be biological, chemical or physical in origin. The skin itself may be the target of these agents or they may be absorbed into the body to cause toxicity in other organs. Dermal toxicology is, therefore, the study of the adverse reactions of the skin resulting from such exposures. Basic research in skin biology provides opportunity for investigation in dermal toxicology. A more complete understanding of the molecular mechanisms underlying the processes involved in the generation of adverse reactions will enable improved models that allow for more accurate predications of potential toxicity as well as provide potential molecular targets for the prevention and treatment of these reactions.

The areas of research encompassed by dermal toxicology include both mechanistic studies of toxicity to the skin itself as well as toxicity to other organs due to absorption through the skin. Of particular interest is the application of these results to the production of risk assessment models for cutaneous exposure to a wide range of potential toxicants, ranging from environmental agents to synthetic chemicals to cosmetics.

U.S. Published Application No. 20180190376 to StoryUp, Inc. discloses the use of VR, AR and/or MR as therapeutic treatment of psychological or psychiatric conditions of a user.

The present invention is directed generally to systems and methods for using virtual reality (“VR”), augmented reality (“AR”) and/or mixed reality (“MR”) in the therapeutic treatment of skin conditions. The present invention is also directed generally to systems and methods for providing specific VR, AR and/or MR content to users as a therapeutic to effect positive changes in the user's biometric data associated with skin conditions.

According to one embodiment, the systems and methods of the present invention may be configured to provide new methodologies implementing VR, AR and/or MR experiences and environments to determine whether the brain can be tricked to perceive “fake” sensation and communicate with distal organs via the Gut-Skin Axis, i.e., whether VR, AR and/or MR can be used to prove the existence of the communication axes between the brain and skin (Brain to Skin Axis [BSA]. This will be conducted through the rendering of a VR, AR and/or MR simulation of skin conditions, including but not limited to, atopic dermatitis, psoriasis, acne, sun burn, scarring, as well as exposure to environmental conditions such as, but not limited to, UV light exposure, pollution, varied climate, etc. Followed by exposing said VR, AR and/or MR rendering to subjects as part of a clinical trial at which biometrics will be collected and analyzed. The systems and methods can then be configured to consistently provide shifts in a user's biometric data toward a desired biometric data pattern (such as EEG brainwave changes, blood pressure changes, heart rate changes, etc.) associated with positive changes in skin condition states of the user.

According to one embodiment, the systems and methods of the present invention may be configured to analyze a user's biometric data. The user's biometric data may be measured during and/or after exposure to certain VR, AR and/or MR content and compared to previously measured biometric data of the user. The results may then be used to identify statistically significant changes in the user's biometric data.

According to one embodiment, the system of the present invention may include a VR headset, a VR content database module, a biometric reference database module, one or more biometric monitors and a processor configured with programming for controlling the operation of the system. The system may be configured to provide selected VR content from the VR content database module to a user or patient through the VR headset. The system may further be configured to measure and record specific biometric data from the user before, during and/or after the selected VR content is provided to the user. The system may further be configured to analyze the recorded biometric data to determine changes in the user's biometric data corresponding to the selected VR content to determine whether the selected VR content provides an effect on the user's skin condition states.

The present invention is also directed to one or more methods for using VR content as therapeutic treatment for a user. According to one embodiment of the present invention, the method may include one or more of the following steps: (i) measuring and recording a user's initial biometrics prior to exposure to selected VR content; (ii) creating a baseline biometric dataset for the user corresponding to the user's initial biometric data; (iii) selecting a first VR content and providing the first VR content to the user to expose the user to a first VR environment and experience; (iv) measuring and recording the user's biometrics during and/or after exposure to the first VR content; (v) creating a first biometric dataset for the user corresponding to the user's biometric data resulting from exposure to the first VR content; and (vi) comparing the user's first biometric dataset with the user's baseline biometric dataset to determine whether the first VR content had a positive therapeutic effect on the user.

The method of the present invention may further be configured to identify changes in the user's biometric data corresponding to the exposure of the first VR content and determine whether the identified changes in biometric data exceed one or more defined threshold requirements. The method of the present invention may further be configured to continue providing the first VR content if the defined threshold requirements are exceeded and configured to modify the first VR content and provide the modified VR content to the user if the defined threshold requirements are not exceeded.

Other aspects and advantages of the present invention will be apparent from the following detailed description of the preferred embodiments of the accompanying drawing figures.

### DESCRIPTION OF THE SEVERAL VIEWS OF THE DRAWING

In the accompanying drawings, which form a part of the specification and are to be read in conjunction therewith in which like reference numerals are used to indicate like or similar parts in the various views:

FIG. 1 is a schematic representation of a system for utilizing virtual reality content to assess the potential impact on skin conditions in accordance with one embodiment of the present invention;

FIG. 2 is a schematic representation and flowchart of a method for utilizing virtual reality content as therapeutic treatment of skin conditions in accordance with another embodiment of the present invention;

FIG. 3 is a schematic representation and flowchart of the method of a FIG. 2 including additional steps in accordance with an embodiment of the present invention;

FIG. 4 is a schematic representation and flowchart of a second method for utilizing virtual reality content as therapeutic treatment of skin conditions in accordance with one embodiment of the present invention.

### DETAILED DESCRIPTION OF THE INVENTION

The invention will now be described with reference to the drawing figures, in which like reference numerals refer to like parts throughout.

The following detailed description of the invention references specific embodiments in which the invention can be practiced. The embodiments are intended to describe aspects of the invention in sufficient detail to enable those skilled in the art to practice the invention. Other embodiments can be utilized and changes can be made without departing from the scope of the present invention. The present invention is defined by the appended claims and the description is, therefore, not to be taken in a limiting sense and shall not limit the scope of equivalents to which such claims are entitled.

### Definitions

“Virtual reality” or (VR) is an interactive computer-generated experience taking place within a simulated environment. It incorporates mainly auditory and visual feedback, but may also allow other types of sensory feedback like haptic. Current VR technology most commonly uses virtual reality headsets or multi-projected environments, sometimes in combination with physical environments or props, to generate realistic images, sounds and other sensations that simulate a user's physical presence in a virtual or imaginary environment. A person using virtual reality equipment is able to “look around” the artificial world, move around in it, and interact with virtual features or items. The effect is commonly created by VR headsets consisting of a head-mounted display with a small screen in front of the eyes, but can also be created through specially designed rooms with multiple large screens. VR systems that include transmission of vibrations and other sensations to the user through devices are known as haptic systems.

“Augmented reality” or (AR) is an interactive experience of a real-world environment where the objects that reside in the real-world are “augmented” by computer-generated perceptual information, sometimes across multiple sensory modalities, including visual, auditory, haptic, somatosensory, and olfactory. The overlaid sensory information can be constructive (i.e., additive to the natural environment) or destructive (i.e., masking of the natural environment) and is seamlessly interwoven with the physical world such that it is perceived as an immersive aspect of the real environment. In this way, augmented reality alters one's ongoing perception of a real world environment, whereas virtual reality completely replaces the user's real world environment with a simulated one. AR is used to enhance natural environments or situations and offer perceptually enriched experiences. With the help of advanced AR technologies (e.g., adding computer vision and object recognition) the information about the surrounding real world of the user becomes interactive and digitally manipulable. Information about the environment and its objects is overlaid on the real world. Augmented reality also has potential in the gathering and sharing of tacit knowledge. Augmentation techniques are typically performed in real time and in context with environmental elements.

“Mixed reality” or (MR) is the merging of real and virtual worlds to produce new environments and visualizations where physical and digital objects co-exist and interact in real time. Mixed reality takes place not only in the physical world or the virtual world, but is a mix of reality and virtual reality.

The distinction between VR, AR and MR are set forth below:
Virtual reality immerses users in a fully artificial digital environment.
Augmented reality overlays virtual objects on the real-world environment.
Mixed reality not just overlays but anchors virtual objects to the real world and allows the user to interact with the virtual objects.

Skin conditions are anything that irritates, clogs, or inflames skin and/or causes symptoms such as redness, swelling, burning, and itching. Allergies, irritants, a person's genetic makeup, and certain diseases and immune system problems can cause rashes, hives, and other skin conditions. Many skin problems, such as acne, can also affect a person's appearance.

“Biometric data” is used herein to refer to biological measurements. Biometric data may include, but not be limited to, brainwaves/brainwave activity, proinflammatory factors, chemoattractant factors, cytokines, chemokines, interleukins, lymphocytes, adhesion molecules, proteases, direct inflammatory cytokines, regulatory cells, pH, skin conductance and lipid concentration.

The present invention is directed generally to systems and methods for using virtual reality (“VR”), augmented reality (“AR”) and/or mixed reality (“MR”) content in the diagnosis and treatment of skin conditions. The present invention is also directed generally to systems and methods for providing specific VR, AR and/or MR content to users to effect positive changes in the user's biometrics associated with skin condition states. The systems and methods of the present invention may be configured to provide VR, AR and/or MR experiences and environments specifically tailored to shift specific biometrics of a user or patient away from patterns associated with negative skin condition states and toward patterns associated with positive skin condition states.

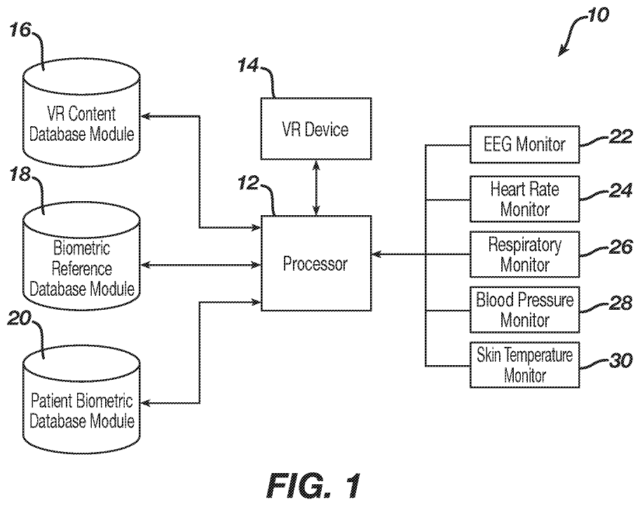

FIG. 1 shows a schematic illustration of a system 10 according to one embodiment configured to provide VR, AR and/or MR content to a patient or user and analyze the patient's or user's biometrics resulting from the provided VR, AR and/or MR content. System 10 may further be configured to be utilized in connection with one or more methods for using VR/AR/MR content in the therapeutic treatment of specified skin conditions as described in greater detail below. The foregoing description of the present invention refers individually to the use of virtual reality (VR) components and concepts in connection with system 10 and methods 100 and 200 disclosed herein; however, it is recognized that augmented reality (AR) and mixed reality (MR) components and concepts may just as suitably be used in place of or in combination with VR components and concepts. As shown in FIG. 1, system 10 may include a processor 12, a VR device 14, a VR content database module 16, a biometric reference database module 18, and/or one or more biometric monitors 22-30 as schematically shown in FIG. 1. As also shown in FIG. 1, system 10 may also include a patient biometric database module 20 for storing biometric data of one or more patients or users of system 10 according to certain embodiments of the present invention.

Processor 12 can be any suitable type of computer processor configured for carrying out one or more sets of programming instructions and sending, receiving, processing and/or storing various types of data and information. According to one embodiment of the present invention, processor 12 may include an application program with programming instructions, that when executed by processor 12 cause the system 10 to carry out one or more steps and/or procedures for sending, receiving, processing and/or storing various types of data and information as described in greater detail below. Processor 12 can be configured to communicate with VR content database module 16 in order to access and transmit VR content based on determined parameters or instructions associated with a patient or user. Processor 12 can be configured to communicate with biometric reference database module 18 in order to access and utilize and biometric data and algorithms for analyzing and processing a patient's biometric data received by system 10. Processor 12 can also be configured to receive and process biometric data from monitors 22-30 and associated with a patient or user.

VR device 14 can be configured as any suitable type of virtual reality device, including but not limited to virtual reality devices commonly known in the art. According to one embodiment, VR device 14 can be configured as a headset that is worn over a user's eyes like a pair of goggles. The headset can block out external light and show a visual representation or image on one or more screens in front of the eyes. The view may be fully or partially immersive, providing a changing field of view in any direction the viewer chooses.

VR content database module 16 can be configured as any suitable storage component and can store a plurality of different VR content accessible by processor 12. Depending on the particular embodiment, VR content database module 16 may comprise a reference library containing a plurality of categorized VR content that is tagged and organized based on content's effectiveness in treatment for certain skin conditions. Database module 16 may further be configured as a searchable database that may be queried by processor 12 to select a desired or appropriate VR content from the plurality of VR content stored in module 16.

Sensors or monitors 22-30 can be configured to monitor, record and/or collect certain types of biometric data of a patient or user before, during and after the user engages with selected VR content through system 10. According to one embodiment, system 10 includes an electroencephalogram (EEG) monitor 22 to monitor the EEG activity of a user. Brain computer interface (BCI) or EEG monitor 22 can be configured as an EEG headband or EEG electrocap that is placed on or around the head of the user of system 10. Depending on the particular embodiment, EEG monitor 22 can be configured to monitor one or more brainwave frequencies, including but not limited to alpha, beta, delta, gamma, and theta, in one or more selected regions of the brain.

System 10 can additionally include a plurality of additional monitors for monitoring and recording additional biometric data of a user in connection with system 10, including but not limited to: a heart rate monitor 24 configured to monitor the heart rate variability of the user; a respiratory sensor 26 configured to monitor the breathing patterns of the user; a blood pressure monitor 28 configured to monitor the blood pressure of the user; and a skin conductance and temperature monitor 30 configured to monitor variations in the external temperature of the user.

System 10 can be used in connection with a user in order to provide VR content from VR content database module 16 and monitor the user's biometric data through monitors 22-30. System 10 can be utilized by fitting a user with VR device 14 and monitors 22-30. Processor 12 may be configured to communicate with VR content database module 16 to receive selected VR content and then transmit the selected VR content to VR device 14. Through VR device 14, the selected VR content can provide a VR environment and experience to the user. Before, during and/or after the selected VR content is provided to the user through VR device 14, processor 12 can be configured to monitor and record the user's biometrics via monitors 22-30 as biometric data. For reference, a user's biometrics as referred to herein can include, but are not limited to EEG readings (including one or more frequencies of brainwaves), heart rate, blood pressure, respiratory patterns, and skin temperature and conductance. Processor 12 may further be configured to analyze the recorded biometric data. According to one embodiment of the present invention, processor 12 may include an application program comprising programming instructions, that when executed by processor 12, cause the system to carry out the steps and procedures above.

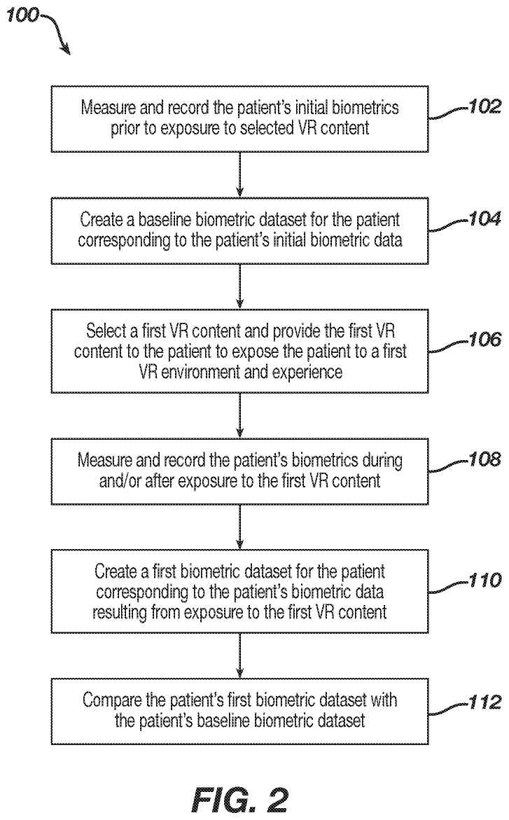

In connection to system 10, the present invention is further directed toward a method 100 for treating a skin condition in a patient through selected VR content. See FIG. 2. Method 100 may be configured to provide targeted VR content to a patient that exposes the patient to a specific VR environment or experience aimed at enacting positive changes in skin condition states as measured through changes in specific biometric parameters of the patient. The method 100 may be further configured to analyze biometric data from the patient to determine whether the provided VR content has the desired effect on the patient as measured by the changes in the patient's biometrics, and/or provide different VR content where the desired effect is not achieved.

As schematically shown in FIG. 2, method 100 may begin at step 102 where the patient's initial biometrics are measured and recorded. As described above, the biometrics recorded for the patient may include EEG readings, heart rate, blood pressure, respiratory rate, and skin temperature, among others. The patient's initial biometric data recorded at step 102 can correspond to the patient's biometric readings prior to exposure to selected VR content. The initial biometric data can measured using monitoring equipment, such as biometric monitors 22-30 of system 10 described above. At step 104, a baseline biometric dataset can be created for the patient based on the initial biometric data recorded at step 102.

After creating the baseline biometric dataset, a first VR content can be selected and provided to the patient at step 106. The first VR content can be provided to the patient through a VR device, such a VR device 14 and can expose the patient to a first VR environment and experience. According to certain embodiments of the present invention, the first VR content is selected based on a selected set of criteria in order to treat a specified skin condition of the patient or cause desired changes in the patient's biometric associated with a specified skin condition state. The VR content can include any number of different components or features, including but not limited to visual stimuli, color, lighting, movement, camera angle, aimed at influencing a patient's skin condition state.

At step 108, the patient's biometrics may be measured and recorded in a similar manner to step 102 during and/or after exposure to the first VR content. During this step 108, depending on the particular embodiment of method 100, the patient's biometrics may be continuously measured or measured at pre-determined intervals or measured only following the completion of the first VR content. At step 110, a first biometric dataset can be created for the patient based on the biometric data measured during step 108 (and corresponding to the patient's exposure to the first VR content).

At step 112, the baseline biometric dataset and the first biometric dataset of the patient may be analyzed to determine the effect the first VR content had on the patient's biometrics associated with skin conditions. According to one embodiment, at step 112, the patient's first biometric dataset is compared to the patient's baseline biometrics to determine variations in the patient's biometric data as described in greater detail below.

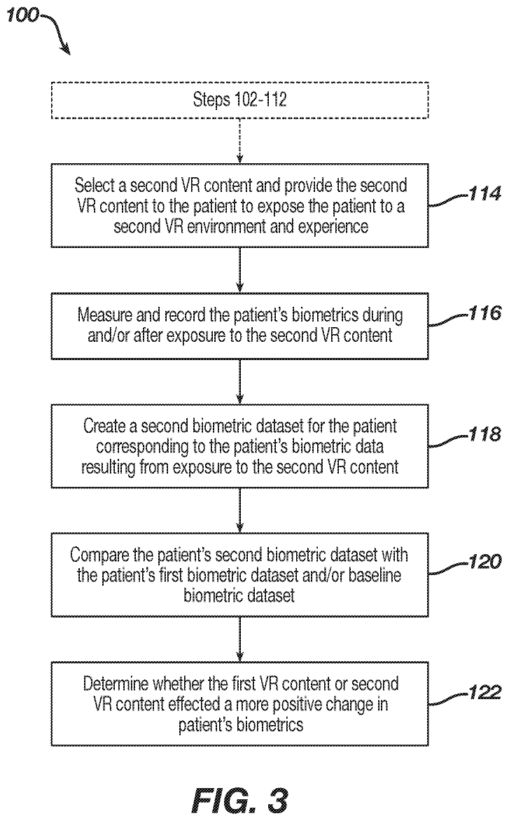

Turning to FIG. 3, in addition to steps 102 through 112, method 100 may include additional steps in certain embodiments of the present invention. As schematically shown in FIG. 3, following step 112, according to one embodiment, method 100 may further comprise selecting and providing a second VR content to the patient to expose the patient to a second VR environment and experience at step 114. At step 116, the patient's biometrics may be measured and recorded during and/or after exposure to the second VR content in a similar manner to steps 102 and 108. At step 118, a second biometric dataset for the patient can be created based on the patient's biometric data measured during step 116. After the second biometric dataset is created, it can be analyzed and compared to the patient's first biometric dataset and/or the patient's baseline biometric dataset at step 120 to determine the effect the second VR content had on the patient's biometrics associated with skin conditions.

A further shown in FIG. 3, according to one embodiment, method 100 may further comprise a step 122 where it is determined, based on the analysis at step 120, whether the second VR content effected a more positive change in the patient's biometrics as compared to the first VR content. The first and second VR contents can be varied by the visual stimuli, color, lighting, movement, camera angle, included within the particular content.

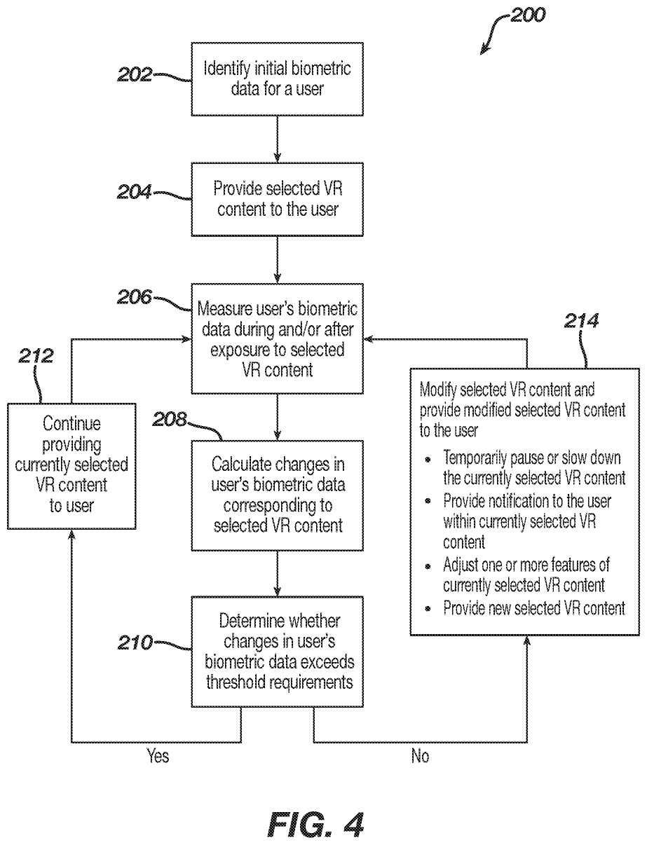

FIG. 4 provides a schematic representation of a second method 200 according to the present invention for providing VR content to a user as therapeutic content. Method 200 can be utilized with system 10 or any other suitable system configured to provide specific VR content to a user and analyze the user's biometrics associated with the specified VR content to effect a positive change in the user's skin condition state. As shown in FIG. 4, method 200 can begin at step 202 by identifying a baseline set of biometric data for a user. The baseline set of biometric data may correspond to the user's initial biometrics prior to exposure to selected VR content as measured and recorded through monitoring equipment or sensors (such as monitors 22-30 in system 10). Following step 202, a selected VR content is provided to the user at step 204 to expose the user to a selected VR experience and environment. The selected VR content may be chosen based on the specific type of skin condition from a library database containing a plurality of VR content categorized based on the content's ability to influence positive change in certain types of skin condition states. The selected VR content may include sound, virtual, augmented or mixed reality experiences, aroma, haptics, audio or other vibration.

As further shown in FIG. 4, at step 206, the user's biometric data is measured and recorded while the user is exposed to the selected VR content and/or after the selected VR content is completed. Depending on the particular embodiment of the present invention, the user's biometric data during step 206 may be recorded continuously or at predetermined intervals. Then at step 208, the changes in the user's biometric data is calculated by comparing the user's biometric data recorded at step 206 with the user's previously recorded biometric data and/or the user's baseline biometric dataset. The calculated changes in the user's biometric data at step 208 are designed to reflect the user's biometric reaction to the currently selected VR content as described in greater detail below.

Then at step 210, it is determined whether the changes in the user's biometric data as calculated at step 208 exceed specific threshold requirements.

As shown in FIG. 4, if it is determined at step 210 that the changes in the user's biometric data corresponding to the currently selected VR content exceed the threshold requirements, then the method proceeds to step 212 and the currently selected VR content continues to be provided to the user. Following step 212, the method loops back to step 206 and the user's biometric data is measured and recorded again during and/or after the selected VR content and steps 206 through 210 are repeated.

If, however, it is determined at step 210 that the changes in the user's biometric data corresponding to the currently selected VR content do not exceed the threshold requirements, the method proceeds to step 214. At step 214, a modified selected VR content is provided to the user in place of the previously selected VR content. The modified selected VR content may comprise one or more of the following modifications, depending on the particular embodiment of the present invention. A notification may be generated and provided to the user within the currently selected VR content.

The currently selected VR content may be replaced with new selected VR content. The new selected VR content may comprise an entirely new or second VR content chosen from a library database containing a plurality of VR content based on the type of skin condition state intended to be addressed for the user.

After providing the modified selected VR content to the user at step 214, the method may then proceed back to step 206 and the user's biometric data may be measured and recorded during and/or after exposure to the modified selected VR content and steps 206 through 210 are repeated.

One objective of method 100 and method 200 of the present invention is to provide new therapeutic methodologies implementing VR experiences and environments tailored to shift a user's brainwaves and other biometrics away from patterns associated with negative skin condition states and toward patterns associated with positive skin condition states. If subjects consistently demonstrate shifts in their biometric data toward the desired biometric data pattern (such as EEG brainwave changes, blood pressure changes, heart rate changes, etc.) and changes in skin condition states consistent with the desired effect, a particular VR content experience will be identified as a targeted therapeutic intervention.

As also described above, the biometrics measured in connection with methods 100 and 200 of the present invention may include EEG (brainwave activity), blood pressure, skin temperature, skin conductance, respiration rate, and heart rate variability. In order to identify positive changes in a user's biometric data corresponding to exposure to certain VR content, the user's biometric data measured before, during and/or after the user is exposed to the VR content may be analyzed. As provided at steps 112 and 120 in method 100 and steps 208 and 210 in method 200 described above, the user's biometric data may be analyzed by comparing biometric data measured during and/or after exposure to VR content with biometric data measured before exposure to the VR content to identify changes in specific types of biometric data. As further provided in method 200 at steps 212-214, when it is determined by analyzing the user's biometric data that the VR content is not providing the desired changes in the user's biometric data, the VR content can be modified or altered to provide a more suitable VR content.

According to one embodiment, the changes identified in the user's biometric data as a result of exposure to selected VR content may be used to determine whether the selected VR content is having a positive effect on the user's skin condition state.

From the foregoing, it will be seen that this invention is one well adapted to attain all the ends and objects hereinabove set forth together with other advantages which are obvious and which are inherent to the structure. It will be understood that certain features and sub combinations are of utility and may be employed without reference to other features and sub combinations. This is contemplated by and is within the scope of the claims. Since many possible embodiments of the invention may be made without departing from the scope thereof, it is also to be understood that all matters herein set forth or shown in the accompanying drawings are to be interpreted as illustrative and not limiting.

The constructions described above and illustrated in the drawings are presented by way of example only and are not intended to limit the concepts and principles of the present invention. Thus, there has been shown and described several embodiments of a novel invention. As is evident from the foregoing description, certain aspects of the present invention are not limited by the particular details of the examples illustrated herein, and it is therefore contemplated that other modifications and applications, or equivalents thereof, will occur to those skilled in the art. The terms “having” and “including” and similar terms as used in the foregoing specification are used in the sense of “optional” or “may include” and not as “required”. Many changes, modifications, variations and other uses and applications of the present construction will, however, become apparent to those skilled in the art after considering the specification and the accompanying drawings. All such changes, modifications, variations and other uses and applications which do not depart from the spirit and scope of the invention are deemed to be covered by the invention which is limited only by the claims which follow.

1. A method for using virtual reality; augmented reality or a combination thereof as therapeutic treatment for a skin condition, said method comprising the steps of: identifying initial biometric data for the user, selecting virtual reality, augmented reality or a combination thereof content for the user based at least in part on skin condition state desired to be improved; providing the selected virtual reality, augmented reality or combination thereof content to the user; measuring the user's biometric data during and/or after the user's exposure to the selected virtual reality, augmented reality or combination thereof content; calculating changes in the user's biometric data corresponding to the selected virtual reality, augmented reality or combination thereof content; determining whether the calculated changes in the user's biometric data exceed a set of threshold requirements; continuing to provide the selected virtual reality, augmented reality or combination thereof content to the user if the calculated changes in the user's biometric data exceed the set of threshold requirements; and
modifying the selected virtual reality, augmented reality or combination thereof content and providing the modified selected virtual reality, augmented reality or combination thereof content to the user if the calculated changes in the user's biometric data do not exceed the set of threshold requirements.

2. The method of claim 1, wherein the step of calculating changes in the user's biometric data includes comparing the EEG brainwave activity of the initial biometric data with the EEG brainwave activity of the measured biometric data.

3. The method of claim 1, wherein the step of modifying the selected virtual reality content includes changing at least one of the visual stimuli, color, lighting, movement, camera angle of the selected virtual reality content.

4. A method for treating a skin condition in a human patient, the method comprising the steps of: measuring the patient's initial biometric activity; creating a baseline dataset corresponding to the patient's initial biometric activity; providing the patient with a first virtual reality content, wherein said first virtual reality content comprises at least one of a virtual reality environment, an augmented reality environment and a mixed reality environment; measuring the patient's biometric activity during and/or after the patient's exposure to the first virtual reality content; creating a first biometric dataset corresponding to the patient's biometric activity resulting from the patient's exposure to the first virtual reality content; and comparing the first biometric dataset with the baseline dataset.

5. The method of claim 4, wherein the patient's biometric activity includes brainwave activity measured using an electroencephalogram (EEG).

6. The method of claim 4, wherein the patient's biometric activity includes at least one of brainwave activity, heart rate, blood pressure, respiratory rate and skin temperature.

7. The method of claim 4, further comprising the step of modifying the patient's brainwave activity by exposing the patient to the first environment.

8. A system for treating a skin condition in a human patient, said system comprising: a processor; a virtual reality content database, said database containing a plurality of different virtual reality content; a virtual reality device configured to provide one or more virtual reality content from said plurality of different virtual reality content to the patient; at least one biometric activity monitor configured to measure the biometric activity of the patient; and an application program comprising programming instructions that, when executed by the processor cause the system to: measure the patient's initial biometric activity using said at least one biometric activity monitor; create a baseline dataset corresponding to the patient's initial biometric activity; provide the patient, through the virtual reality device, with a first virtual reality content from the plurality of different virtual reality content, wherein said first virtual reality content comprises at least one of a virtual reality environment, an augmented reality environment and a mixed reality environment; measure the patient's biometric activity during and/or after the patient's exposure to the first virtual reality content using the at least one biometric activity monitor; create a first biometric dataset corresponding to the patient's biometric activity resulting from the patient's exposure to the first virtual reality content; and
compare the first biometric dataset with the baseline dataset.

9. The system of claim 8, wherein the patient's biometric activity includes brainwave activity measured using an electroencephalogram (EEG).

10. The system of claim 8, wherein the patient's biometric activity includes at least one of brainwave activity, heart rate, blood pressure, respiratory rate and skin temperature.

11. The system of claim 8, wherein the application program further comprises programming instructions that, when executed by the processor cause the system to: provide the patient, through the virtual reality device, with a second virtual reality content from the plurality of different virtual reality content, wherein said second virtual reality content comprises at least one of a virtual reality environment, an augmented reality environment and a mixed reality environment; measure the patient's biometric activity during and/or after the patient's exposure to the second virtual reality content using the at least one biometric activity monitor; create a second biometric dataset corresponding to the patient's biometric activity resulting from the patient's exposure to the second virtual reality content; and compare the second biometric dataset with at least one of the first biometric dataset and the baseline dataset.
